(19) United States
(12) Patent Application Publication
(10) Pub. No.: US 2007/0128244 A1
(43) Pub. Date: Jun. 7, 2007

(54) Bioceramic scaffolds for tissue engineering

(76) Inventor: Stuart K. J. Smyth, Shelburne, VT (US)

Correspondence Address: STUART K. J. SMYTH, P.O. BOX 1059, Shelburne, VT, 05482, US

(21) Appl. No.: 11/293,707

(22) Filed: Dec. 5, 2005

(51) Int. Cl. A61F 2/02

(52) U.S. Cl. 424/423; 977/906

(57) ABSTRACT

This invention relates to a porous ceramic sphere comprising a particulate micro-porous bioinert material, in the form of spheres or scaffolds, having interconnected pores. Pores on the surface of each sphere are connected to pores inside such sphere via blow-holes and the internal pores are in turn interconnected, so that in addition to a high porosity, the spheres have a high permeability to gas and liquid, thus contributing to the creation of an artificial vascular system in which cells will proliferate, migrate and differentiate into the specific tissue while secreting the extracellular matrix components required to create the 3D tissue and organs.

### INTRODUCTION

This invention relates to porous ceramic scaffolds for tissue engineering.

More particularly, the invention relates to the method and means to produce a supply of immunologically tolerant “artificial” organ and tissue substitutes that can grow with the patient.

More specifically, but not exclusively, the invention relates to porous ceramic scaffolds that lead to a permanent solution to damaged organs or tissues with or without the incorporation of supplementary therapies or pharmaceutical preparations.

### BACKGROUND TO THE INVENTION

Tissue engineering is a new and exciting technology that has the potential to create tissues and organs de novo. It involves the in vitro seeding and attachment of human cells onto a scaffold. Once implanted, the cells proliferate, migrate and differentiate into the specific tissue while secreting the extracellular matrix components required to create the tissue. The choice of scaffold is crucial to enable the cells to behave in the required manner to produce the tissue and organs of the desired shape and size. Current scaffolds, made by conventional scaffold fabrication techniques, are generally foams of synthetic polymers. The cells do not necessarily recognize such surfaces, and most importantly cells cannot penetrate more than 500 micrometers into the scaffold. The lack of oxygen and nutrient supply governs this depth.

One of the principal methods used in tissue engineering involves growing the relevant cell(s) in vitro into the required three-dimensional (3D) organ or tissue. Cells lack the natural ability to grow in favoured 3D orientations and thus defme the anatomical shape of the tissue. Instead, they randomly migrate to form a (2D) layer of cells. However, 3D tissues are required and this is achieved by seeding the cells onto porous matrices, known as scaffolds, to which the cells attach and colonize. The scaffold therefore is a very important component in tissue engineering.

Investigations into synthetic and natural inorganic ceramic materials, for example hydroxyapatite and tri-calcium phosphate, as candidate scaffold materials, have been aimed mostly at bone tissue engineering. This is primarily because these ceramics simulate the natural composition of bone and have osteoconductive properties. Other examples of materials utilized for scaffolding are the synthetic polymers. However, the degradation of synthetic polymers, both in vitro and in vivo, releases acidic by-products which raises concerns that the scaffold microenvironment may not be ideal for tissue growth. Lactic acid is released from PLLA during degradation, reducing the PH, which further accelerates the degradation rate due to autocatalysis, resulting in a highly acidic environment adjacent to the polymer. Such an environment may adversely affect cellular function. Cells attached to scaffolds are faced with several weeks of in vitro culturing before the tissue is suitable for implantation. Moreover, current synthetic polymers do not possess a surface chemistry which is familiar to cells that in vivo thrive on extracellular matrix made worthy of collagen, elastin, glycoproteins, proteoglycans, laminin and fibronectin. In contrast, collagen is the major protein constituent of the extracellular matrix and is recognized by cells as being chemostatic. Collagen scaffolds may present a more native surface relative to synthetic polymer scaffolds for tissue engineering purposes. However, like other natural polymers, collagen may elicit an immune response.

Most conventional scaffolding fabrication techniques are incapable of precisely controlling pore size, pore geometry, spatial distribution of pores and construction of internal channels within the scaffold. Typically, scaffolds produced by solvent-casting particulate leaching cannot guarantee interconnection of pores because this is dependent on whether the adjacent salt particles are in contact. Furthermore, skin layers that are formed during evaporation and agglomeration of salt particles make controlling the pore sizes difficult. For gas foaming, it has been reported that 10-30% of pores are interconnected. Non-woven fibre meshes have poor mechanical integrity.

Excluding gas foaming and salt molding, conventional scaffold fabrication techniques use organic solvents like chloroform and methylene chloride to dissolve synthetic polymers at some stage in the process. The presence of residual organic solvent is the most significant problem facing these techniques due to the risks of toxicity and carcinogenicity it poses to cells.

Conventional fabrication techniques produce scaffolds that are foam structures. Cells are then seeded and expected to grow into the scaffold. However, this approach has resulted in the in vitro growth of tissue with cross sections of less than 500 micrometers from the external surface. This may be due to the diffusion constraints in the foam. The pioneering cells cannot migrate deep into the scaffolding because the lack of nutrients and oxygen. The insufficient removal of the waste products that cell colonization at the scaffold periphery consumes also causes an effective barrier to the diffusion of oxygen and nutrients into the interior of the scaffold. Thus cells are only able to survive close to the surface. In this connection, it should be noted that no cell, except for chondrocytes, exists further than 25-100 micrometers away from a blood supply. The low oxygen requirement for cartilage may be the reason why only this tissue has been successfully grown in vitro to thick cross-sections i.e., greater than 1 mm using conventional scaffold fabrication techniques. Skin is a relatively 2D tissue and thus thick cross-sections of tissue are not required, thereby explaining the success of producing this tissue with conventional scaffold fabrication techniques. However, most other 3D tissues require a high oxygen and nutrient concentration.

Because of the limitations of current scaffolding materials, there exists the problem of providing a bioactive, bioinert scaffold that will have some form of an artificial vascular system present to increase the mass transport of oxygen and nutrients deep within, and the removal of waste products from the scaffold. There is a demonstrated need for a scaffold that would permit pioneering cells to migrate deep into the scaffold and which may host other supplementary therapies or materials.

### OBJECTS OF THE INVENTION

It is therefore an object of the present invention to provide bioinert, but activated porous ceramic scaffolds that can provide the optimum conditions suitable for an artificial vascular system for the seeding and development of pioneering cells that form into a desired shape and size. It is also within the same object of the present invention to create scaffolds which form an artificial vascular system, sustainable both in vitro and in vivo; which will host the seeding and attachment of human cells, and continuously provide oxygen, nutrients and waste removal which will then cause the cells to proliferate, migrate and differentiate into specific tissue while secreting the extracellular matrix components required to create the tissue.

### SUMMARY OF THE INVENTION

According to the invention, ceramic spheres with engineered porosities that extend their functionality, purpose, design and properties beyond that of just a ceramic sphere, but which may be defined as the component(s) necessary to temporarily or permanently establish an environment for the creation of an artificial vascular system which in turn may ultimately create the host environment for the proliferation, migration and differentiation into of cells into 3D tissue.

According to the invention there is provided tissue scaffolds, which may comprise a particulate micro-porous, bioinert ceramic material, having interconnected pores.

Further, according to the invention the particles may be in the form of spheres.

Further, according to the invention the micro-pores may be exposed on the surface of the spheres, hence rendering the surface of the spheres smooth on a macro-scale, but porous and uneven on a micro-scale in order to enhance and promote the seeding and attachment of human cells into and onto the scaffolds.

Yet further, according to the invention the pores on the surface of each sphere may be connected to the pores inside the sphere via blow-holes and these internal pores are in turn interconnected, so that in addition to a high porosity, the spheres have a high permeability to gas and liquid such as oxygen and blood which are vital to the survival of the cells.

Yet further to the invention, the macroarchitecture of the scaffold spheres may be reliant upon the microarchitecture of the spheres in order to host cells and thereby complete the intended outcome of tissue engineering.

Yet further to the invention, the microarchitecture of the scaffold spheres may be reliant on the environment, properties and design, which are provided due to the macroarchitecture of each of the scaffold spheres.

Yet further to the invention, are spheres which may comprise the intrinsic and extrinsic properties of bioceramic scaffolds that may be milled or manufactured utilizing various methods or means; including computerized assistance or other imaging techniques, which may create the necessary porosity, size, strength, surface smoothness, fragility, time frame of usage, absorbability, construction of the internal channels and pore geometry in tissue engineering.

Yet further to the invention, the microarchitecture and the macroarchitecture of each sphere may not be able to complement one another without the reliance upon the design and properties of both the intrinsic properties of each sphere and the extrinsic properties and without the reliance in design and properties, the spheres cannot provide a scaffold for cells from which the cells may proliferate, migrate and differentiate into specific tissue while secreting the extracellular matrix components required to create tissues and organs de novo. For without the porous microarchitecture and varying degree of strength of each sphere, the ceramic microspheres may only provide limited or negligible functionality and thereby not achieve the desired intention of the invention.

Yet further to the invention, a porous ceramic scaffold with interconnected pores whereby the microarchitecture of each sphere comprising the scaffolds, may temporarily or permanently prevent the exterior of the sphere from acting as an effective barrier to the diffusion of oxygen and nutrients and waste removal, or may until such time act as an effective barrier until the cells have reached a certain degree of growth and have begun to propagate, migrate and differentiate.

Yet further to the invention, a scaffold that may not limit access of cells to the microarchitecture, which may only be accessible by the chondrocytes which are typically able to survive 25-100 micrometers from the blood supply.

The spheres which comprise the scaffolds may have a high permeability to gas and liquid and may provide waste removal, thereby creating an artificial vascular system for the mass transport of oxygen and nutrients deep within the microarchitecture of the scaffolds, as well as the necessary removal of waste products from the scaffolds.

A scaffold that promotes 3D or 2D formation of cellular structures because of the macroarchitecture and the microarchitecture of the scaffolds.

The spheres, which comprise the scaffold, may have a varying or consistent porosity of between 30% and 70% per volume.

The pores of the spheres, which comprise the scaffolds, may have diameters in the range of from 0.3 to 15 micrometers.

The size of the spheres may vary between 100 to 600 microns, but may be smaller or larger dependent upon the intended tissue application.

The spheres which comprise the scaffold may have a diameter larger than 100 micrometers, and may be larger or smaller should the need arise however; the diameter of the spheres may not be below 45 micrometers.

The scaffolds may permit within the microarchitecture, the implantation and activation of alternative or complimentary nanotechnologies: examples may include certain medical devices, semiconductors, trace elements, mediums, minerals, chemicals, electromagnetic activities, light transmission activity, ultrasonic activity and magnetic activity. The incorporation of nanotechnologies when combined with the scaffold may lead to an interconnected relationship between the spheres and scaffold's complete architecture both in vitro and in vivo. Such an in vivo relationship may be interconnected in some function, purpose or properties to that of a diagnostic, therapeutic or management aspect of tissue via both internal and external elements, force and energy.

The scaffolds may attract, sustain or cooperate with a force of energy, which acts to aid in the growth, or control of the cellular activity. Such energy contained within the scaffold's architecture may pass through the architecture or it may be stored deep within the architecture in order to be activated for some intended purpose.

Scaffolds, comprised of porous bioceramic spheres, which may be controlled both in vivo and extra corporal cellular or tissue activities/responses such as rate and/or time of cell/tissue growth, size, dimension, volume, weight, density, fluidity, viscosity, acidity, alkalinity, absorption, waste removal and saturation.

The bioinert spheres, which comprise the scaffolding, may be selected from a group of ceramic materials consisting of sintered aluminium oxide (alumina); sintered zirconium oxide (zirconia); combinations thereof or hydroxyapatite.

The spheres may be activated with the cells prior to in vitro seeding and prior to the introduction of the scaffolds into the body. The scaffolds may thereby be bioactive and “Biospecific” to each individual patient prior to their actual injection into the specific patient.

The spheres may be activated by the in situ impregnation with serum from the blood of a patient thereby rendering the scaffold as “Biospecific” and therefore completely biocompatible.

The porous spheres, which comprise the scaffolding, may be mobilized for injection by mixing the spheres with a biosuitable carrier gel or a viscous, lubricating carrier liquid. However, the serum of a patient's blood may prove suitable alone as a carrier, without the need for an additional gel or carrier liquid. The fmal transportation of such unaided fluids may be aided by an external force of energy in order to prevent bridging or clogging during the process of injection, and an external force may prove beneficial in ensuring the impregnation of the serum of the patients own blood within the microarchitecture and surrounding the macroarchitecture of each sphere prior to or during the seeding process.

Precipitating a resorbable calcium phosphate into the microarchitecture of the pores may also activate the spheres, which comprise the scaffolds.

The porous scaffolds may permit the usage of supplementary therapies and may act as temporary carriers, receptors or chemo attractors for a desired population of cells.

The scaffold may provide osteoconductive properties, which may or may not be blended into a polymer matrix or cement-like material.

Another aspect of the invention is a scaffold that creates an environment for syneresis of the tissues.

Another aspect of the invention is a scaffold that helps to change the desired population of particular targeted cells, which in turn provides an artificial vascular system for the growth of new cell populations.

Another aspect of the invention is a scaffolding to create tissues.

Another aspect of the invention is providing a scaffolding material to create organs.

Another aspect of the invention is to provide a scaffolding material that may be utilized for targeted drug delivery both during the process of seeding and the post-seeding period of the growth of cells and tissues.

Another aspect of the invention is a scaffold, which may be used concomitantly with other surgical therapies such as tissue sealants or tissue adhesives and because of the properties of the scaffold, the scaffolds aid in the tissue healing process.

The scaffolding, according to the present invention is prepared, but not limited too; a method including the steps of:
Milling the pure precursor ceramic raw material into powder with particle size finer than 1 micrometer.
Blending a combustible substance known in the trade of manufacturing porous structures into powder
Mixing the powder with water to form a paste or slurry
Formning solid spherical particles from the slurry by applying methods known in the art
Sintering the spherical particles at a temperature between 1350 C and 1650 C to form inert micro-porous ceramic spheres.having pore sizes of between 0.3 and 15 micrometers and a diameter of between 100 to 600 micrometers but not smaller than 45 micrometers, and, screening the spheres into pre-selected, preferably, but not exclusively, narrow size fractions, for example 45 to 100 micrometers, 100 to 200 micrometers, 200 to 300 micrometers, 300 to 400 micrometers and so forth.

The applicants have found according to the present invention that scaffolding that meets most of the requirements for cells, once seeded, to proliferate, migrate and differentiate into specific tissues while secreting extracellular matrix components required to create tissue and organs, have the following properties in common:
Possess interconnecting pores of an appropriate scale to form tissue integration and vascularization
Have appropriate surface chemistry to favour cellular attachment, differentiation and proliferation
Possess adequate mechanical properties to match the intended site of implantation and handling
Should provide varying degrees of strength to achieve the desired outcome
Does not induce any adverse response
Is not immunogenic, hypoallergenic and nonantigenic
Is biocompatible and bioinert, but biologically activated
Immobile
No measurable inflammatory response
Includes spheres having diameters larger than 100 micrometers, typically 110 to 600 micrometers, being larger or smaller as the need should rise, but the spheres should not have diameters less than 45 micrometers
Provides synthesis in functionality and purpose between the macroarchitecture and the microarchitecture of the spheres, which in turn comprise the scaffolds once activated with serum or other carrier liquids/gels which ultimately hosts the environment to create an artificial vascular system that will host the cells in order to aid the cells in the process of proliferation, migration and differentiation into the desired tissues or organs.

The description of the embodiments of the present invention is given above for the understanding of the present invention. It is appreciated further that the invention is not limited to the particular embodiments described herein, but is capable of various modifications and rearrangements as will be apparent to those skilled in the art without departing from the scope of the appended claims.

What is claimed is:

1) A porous ceramic scaffold (sphere) with an engineered porosity that extends functionality, purpose and properties beyond that ofjust a typical ceramic sphere.

2) A porous ceramic scaffold, which according to claims 1 wherein the porous spheres provide a temporary environment for the creation of an artificial vascular system. A sphere that provides a temporary environment refers to a ceramic sphere fabricated with a specific dimension, strength, and degree of fragility, tolerance and porosity.

3) A porous ceramic scaffold, which according to claims 1-2 wherein the temporary sphere or scaffold bursts, shatters, breaks, splinters, shreds, splits, dissolves or becomes altered in any way from its original state when it was first introduced into the body and prepared ex vivo. The action of bursting, shattering, breaking, splintering, shredding, splitting or becoming altered in any way from when it was first introduced into the body will create ceramic microfragments that will not migrate because they too are porous, bioinert and will remain imbedded in tissue without impeding the further development of cellular activities.

4) A porous ceramic scaffold, which according to claims 1-3 wherein the porous spheres create a host environment for the seeding deep within the microarchitecture of the scaffold for the proliferation, migration and differentiation of cells.

5) A porous ceramic scaffold, which according to claims 1-4 wherein the scaffold is comprised of micro-porous, bioinert ceramic material, having either even or uneven interconnected pores.

6) A porous ceramic scaffold, which according to claims 1-5 are exposed on the surface of the spheres, hence rendering the surface of the spheres smooth on a macro-scale, but porous and uneven on a micro-scale thereby enhancing and promoting the seeding of cells and subsequent attachment of human cells into the sphere, through the sphere and onto the surface of the ceramic scaffold material.

7) A porous ceramic scaffold, which according to claims 1-6 wherein the pores on the surface of each sphere are connected to at least some of the pores inside the spheres via blow-holes and these either even or uneven internal pores are interconnected, so that in addition to a high porosity, the spheres have a high permeability to gas and liquid such as oxygen and blood which are necessary for the development of cells, tissue and organs.

8) A porous ceramic scaffold, which according to claims 1-7 wherein the functionality and outcome of the scaffold is a result of the codependent or combined relationship between the individual properties of the microarchitecture properties in combination with the macroarchitecture of the scaffold, and as a result of the properties of the entire scaffold, the scaffold will host the environment for an artificial vascular system thereby differentiating the porous ceramic sphere from a typical ceramic sphere which is incapable of providing an artificial vascular system because it lack the properties of both the microarchitecture and macroarchitecture and their combined effects.

9) A porous ceramic scaffold according to claims 1-8 wherein by fabricating a scaffold that creates an environment for the proliferation, migration and differentiation of cells; the scaffold is comprised of codependent and interdependent intrinsic and extrinsic properties in order to achieve a viable scaffold material which is designed to provide a desired outcome in tissue engineering, and which are milled or fabricated utilizing a computer or other imaging device to achieve the desired porosity, size of pores, strength of pores, degree of fragility, surface smoothness, construction of internal channels and pore geometry applicable in tissue engineering.

10) A porous ceramic scaffold according to claims 1-9 wherein the strength and/or degree of fragility is a direct result of the porosity of 30% and 70% per volume depending upon the tissue engineering required.

11) A porous ceramic scaffold according to the claims 1-10 wherein the porosity, strength and/or degree of fragility of each sphere provides for the necessary conditions to host an artificial vascular system. As the necessary conditions are created for the proliferation, migration and differentiation of cells, during the earlier stage of the cellular activity, the porous ceramic sphere can be fabricated to be brittle or fragile enough so as to eventually, fragment, burst, crack, shatter, break, splinter, shred or change form so as to provide a means for the cells to proliferate, migrate and differentiate into 3D tissue. The microfragments or residual pieces of the ceramic scaffold will not impede further cellular activity, ability for the tissue to grow into a desired tissue-engineering outcome, and the microfragments will in fact render themselves obsolete or may dissolve over time with the aid of biocompatible corrosive chemical materials. A comparison of this may be derived by way of comparing and contrasting the porous ceramic microfragments to that of the shell of a chicken's egg. That is, the fertilized egg may be compared to the cells which grow into a particular fetal form, which then turn into a chick- all of this is possible under the right growing conditions for any healthy egg. Once the chick is ready to hatch, it cracks the shell with its beak and applies force so as to escape the shell of the egg altogether. Once this is achieved, the chick may eventually grow into a healthy adult chicken.

12) A porous ceramic sphere, which according to claims 1-11 wherein the porous ceramic scaffolds are constructed for the removal of waste products that cell colonization, normally removes at the periphery. Thus, a porous ceramic scaffold that is deigned to remove products that are barriers to the diffusion of oxygen and nutrients, and thereby results in a scaffold that creates an artificial vascular system for the proliferation, migration and diffusion of cells into 3D tissues.

13) A porous ceramic sphere, which according to the claims in 1-12 wherein a scaffold structure that readily permits cells to enter deep into the microarchitecture of each sphere, and thus results in seeding of various living cells, which are not exclusively chondrocytes- and which typically survive 25-100 micrometers from a blood supply-to enter deep within the microarchitecture of each sphere and remain nourished and viable because of the properties of the microarchitecture of the scaffold that provides an artificial vascular system.

14) A porous ceramic sphere, which according to claims 1-13 wherein the scaffolds have diameters in the range of 0.3 to 15 micrometers.

15) A porous ceramic sphere which according to claims 1-14 wherein the diameters of the ceramic spheres vary between 100 to 600 microns but may be smaller or larger depending upon the desired outcome for tissue engineering.

16) A porous ceramic sphere, which according to claims 1-15 wherein the combined size of both the ceramic sphere's microarchitecture and macroarchitecture permit concomitant implantation, application and utilization of nanotechnologies; nanotechnologies such as semiconductors, microtransmitters, trace element nanotechnologies, medium based nanotechnologies, mineral based nanotechnologies, chemical based nanotechnologies, thermal based nanotechnologies, light transmission based nanotechnologies, nanoreceptors, biosensors, pressure transducer nanotechnologies, electromagnetic nanotechnologies, radiofrequency nanotechnologies, ultrasound based nanotechnologies, ultrasound based nanotechnologies, nuclear based nanotechnologies, magnetic induced nanotechnologies, ferromagnetic fluid based nanotechnologies, video and photodynamic nanotechnologies, diagnostic nanotechnologies,therapeutic nanotechnologies, gas reactive nanotechnologies, and energy reactive nanotechnologies.

17) A porous ceramic sphere, which according to claims 1-16 wherein spheres will attract, absorb, sustain and release certain nanotechnology substances or nanotechnology energy based byproducts deep within the microarchitecture of the porous sphere; and such energy based nanotechnology byproducts or substances may be transmitted or retransmitted, as a result of the porous microarchitecture of the scaffold, back to the origin of the original source of nanotechnology substances or various sources of energy -and the byproducts or substances may be transmitted, because of the porous microarchitecture of the scaffold, therefore permit the targeting of certain tissues and organs within the human body.

18) A porous ceramic sphere, which according to claims 1-17 wherein bioinert spheres may be selected from a group consisting of sintered aluminum oxide (alumina); sintered zirconium oxide (zirconia); or combination thereof.

19) A porous ceramic sphere which according to claims 1-18 wherein the combined properties of the microarchitecture and the macroarchitecture of the scaffold permits the incorporation of certain pharmaceutical preparations, and thus the scaffolds become receptors or chemoattractors for a desired population of cells. The scaffolds will then serve to aid in the advancement of cellular activity, provide an arena for syneresis of the tissue and, if required, will erode particular targeted cells that will be replaced by healthy cells.

20) A porous ceramic sphere, which according to claims 1-19 wherein the ceramic spheres are osteoconductive and which may be blended with polymatrix or cement-like material and with tissue sealants or tissue adhesives made of polymers and hydrogels. The scaffold is mixed with the tissue sealants or tissue adhesives.
